(19) United States
(12) Patent Application Publication
(10) Pub. No.: US 2009/0131359 A1
(43) Pub. Date: May 21, 2009

(54) ANTIFIBROTIC THERAPY

(76) Inventors: Sergei P. Atamas, Ellicot City, MD (US); Irina G. Luzina, Ellicott City, MD (US); Nevins W. Todd, Baltimore, MD (US)

Correspondence Address: MORGAN LEWIS & BOCKIUS LLP, 1111 PENNSYLVANIA AVENUE NW, WASHINGTON, DC, 20004, US

(21) Appl. No.: 12/273,149

(22) Filed: Nov. 18, 2008

Related U.S. Application Data
(60) Provisional application No. 60/989,076, filed on Nov. 19, 2007.

(51) Int. Cl. A61K 48/00; C12Q 1/02

(52) U.S. Cl. 514/44; 435/29

(57) ABSTRACT

The present invention relates to methods of treating severe or rapidly progressing pulmonary fibrosis in a subject in need of a treatment thereof. The methods comprise increasing the activity of pulmonary and activation-regulated chemokine (CCL18) in the lungs of the subject, whereby increasing CCL18 activity modulates the activity of at least one antifibrotic factor in the lungs of the subject. The present invention also relates to methods of screening test procedures that may be capable of treating severe or rapidly progressing pulmonary fibrosis.

### CROSS-REFERENCE TO RELATED APPLICATIONS

This application claims priority to U.S. Provisional Application No. 60/989,076, filed 19 Nov. 2007, which is incorporated by reference.

### STATEMENT REGARDING FEDERALLY SPONSORED RESEARCH OR DEVELOPMENT

Research performed during development of this invention utilized U.S. Government funds from NIH Grant Number HL074067 and VA Merit Review Type I Award. The U.S. Government has certain rights in this invention.

### BACKGROUND OF THE INVENTION

1. Field of the Invention

The present invention relates to methods of treating severe or rapidly progressing pulmonary fibrosis in a subject in need of a treatment thereof. The methods comprise increasing the activity of pulmonary and activation-regulated chemokine (CCL18) in the lungs of the subject; whereby increasing CCL18 activity modulates the activity of at least one antifibrotic factor in the lungs of the subject.

In particular, the present invention relates to methods of treating, preventing, or preventing the progression of pulmonary fibrosis.

2. Background of the Invention

Pulmonary fibrosis is usually diagnosed by a history of progressive shortness of breath with exertion, crackling sounds in the chest by stethoscope examination, abnormal CAT scan, and/or abnormal lung functioning test. The diagnosis may also be confirmed by lung biopsy in which the chest wall is surgically opened under general anesthesia to remove a portion of lung tissue, and the removed tissue is examined microscopically to confirm the presence of fibrosis. Schiffman G., Pulmonary Fibrosis, Medicinenet.com (Stoppler M.C., ed., last editorial review Dec. 6, 2006).

The treatment for idiopathic pulmonary fibrosis is very limited, and there is presently no evidence that any medication(s) can treat this condition, since scarring is usually permanent throughout the lungs. Currently, lung transplantation is the only therapeutic option available. Studies exploring drug treatment to reduce fibrous scarring are ongoing. For example, because the immune system is centrally implicated in the development of pulmonary fibrosis, corticosteroids (e.g., prednisone) is used to suppress the immune system in order to attenuate physiological processes that lead to fibrosis, e.g., to decrease lung inflammation and subsequent scarring. However, the side effects and toxicity of drug treatments may be serious. In this regard, only a minority of patients respond to corticosteroids alone, so additional immunosuppressive medications are used in conjunction with corticosteroids. These drugs include gamma-interferon, cyclophosphamide, azathioprine, methotrexate, penicillamine, cyclosporine, and anti-inflammatory medications, but have met with limited success.

Patients with pulmonary fibrosis are subjected to a vicious cycle of oxygen deprivation that requires supplemental oxygen treatment to prevent pulmonary hypertension. This type of hypertension is caused by an initial decrease in blood oxygen levels that creates a hypoxic situation that leads to the pulmonary hypertension from the elevated pressure in the pulmonary artery. This pulmonary hypertension eventually leads to failure of the right ventricle of the heart. Schiffman G., Pulmonary Fibrosis, Medicinenet.com (Stoppler M.C., ed., last editorial review Dec. 6, 2006).

The present inventors have recently reported that levels of a CC chemokine CCL18 mRNA and protein are increased in alveolar macrophages and bronchoalveolar lavage (BAL) fluid, respectively, in patients with scleroderma lung disease (1), a condition characterized by the accumulation of T cells in the lungs and by pulmonary fibrosis (2). Increases in CCL18 have also been reported in the lungs of patients with other pulmonary diseases characterized by T cell involvement and collagen deposition, such as hypersensitivity pneumonitis and idiopathic pulmonary fibrosis (3,4), pulmonary sarcoidosis (5), and allergic asthma (6,7).

CCL18, also known as pulmonary and activation-regulated chemokine (PARC), macrophage inflammatory protein 4 (MIP-4), alternative macrophage activation-associated CC chemokine 1 (AMAC-1), dendritic cell-derived chemokine1 (DCCK1), and small secreted cytokine A 18 (SCYA-18), is constitutively expressed at high levels in the lungs (3,8-10) and is selectively chemotactic for T cells (11).

The present inventors have found (12-14), and others have recently confirmed (4) that CCL18 in high concentrations (300-1000 ng/ml) acts directly on cultured primary pulmonary fibroblasts, activates intracellular signaling, and stimulates collagen production in a time- and dose-dependent manner.

Macrophages produce CCL18 alternative activation in a Th2 environment (8,15-17) and directly stimulate collagen production in fibroblasts (18). In vivo, CCL18 may promote pulmonary fibrosis when expressed at much lower concentrations (300 pg/m), by attracting T cells to the lungs (11).

T cells constitute a relatively minor population in a normal lung, although the T cell population expands numerically and undergoes phenotypic changes in association with lung inflammation and fibrosis (2,19-22). Although not all pulmonary fibrotic processes are T cell lymphocyte-dependent (23-25), previous studies suggest that T lymphocytes contribute to regulation of fibrosis in the lung in human disease (1,2,19-21) and in animal models of pulmonary fibrosis (22,26,27).

In contrast to pulmonary T lymphocytes, macrophages are by far the most abundant cell type in the lungs, normally constituting more than 85% of bronchoalveolar lavage cells (2). Although the percentage of macrophages declines during lung inflammation due to influx of T cells and other inflammatory cells, macrophages undergo phenotypic changes associated with inflammation and fibrosis (1), and establish a vicious circle of pulmonary fibrosis by further upregulating CCL18 expression (4).

The present inventors have developed a CCL18 overexpression animal model that resembles human pulmonary fibrotic disease. The animal model manifests pulmonary T lymphocytic infiltration, TGF-βactivation, and T cell-dependent collagen accumulation (11). However, in humans with pulmonary fibrosis, the elevation of pulmonary levels of CCL18 occurs in the context of pulmonary inflammation and fibrosis that may affect the outcome of CCL18 expression in the lungs. Elevated pulmonary levels of CCL18 have been associated with influx of T lymphocytes, collagen accumulation, and a decline in lung function in pulmonary fibrosis patients. It was previously reported that overexpression of CCL18 in mouse lungs triggers selective infiltration of T lymphocytes and moderate lymphocyte-dependent collagen accumulation. Considering that both CCL18 expression (11) and inflammation (28) are profibrotic, it was hypothesized that the combined action of CCL18 overexpression and bleomycin-induced lung injury would cause a profound, additive or synergistic, fibrotic lung damage.

The present inventors report here that unexpectedly, increasing CCL18 activity in the lungs of an animal with severe or rapidly progressing pulmonary fibrosis is actually protective against chemical-induced injured. The mechanisms of this phenomenon are addressed by following the changes in the levels of the factors that known to be involved in the regulation of fibrosis, including matrix metalloproteinases MMP2 and MMP9 and cytokines TGF-β, IL-13, TNF-α, and IFN-γ (29-33).

The present inventors have also found that the CCL8-attracted pulmonary T cell lymphocytes act profibrotically in otherwise healthy lungs but partially antifibrotically in the presence of a profibrotic injury (induced by a drug such as bleomycin). The implication of this observation is that a therapeutic elimination of T cell lymphocytes from the inflamed lungs may have a counterintuitive, deleterious effect in the patient. Furthermore, there may be potential for further enhancing the antifibrotic regulation in the lungs by therapeutically manipulating the local pulmonary milieu and/or the phenotypes of infiltrating T lymphocytes.

The present inventors infected mice with a replication-deficient adenovirus encoding CCL18 and instilled a drug that induces pulmonary injury (e.g., bleomycin). In addition, control mice were challenged with either CCL18 overexpression or at least one drug that induces pulmonary injury such as inflammation. The additive effects of CCL18 overexpression and drug-induced injury were observed on pulmonary inflammation, particularly on T cell infiltration, and increased levels of TNF-α, IFN-γ, MMP2, and MMP9.

Despite the hypothesis that CCL18 would have additive effect on inflammation, the present inventors found that CCL18 overexpression unexpectedly attenuated the drug-induced collagen accumulation. Pulmonary levels of active TGF-β mirrored the changes in collagen levels. Depletion of T cells with anti-lymphocyte serum or pharmacological inhibition of MMPs with GM6001 abrogated accumulation of collagen and increases in the levels of TNF-α, IFN-γ, and active TGF-β.

### SUMMARY OF THE INVENTION

The present invention relates to methods of treating severe or rapidly progressing pulmonary fibrosis in a subject in need of a treatment thereof. The methods comprise increasing the activity of pulmonary and activation-regulated chemokine (CCL18) in the lungs of the subject, whereby increasing CCL18 activity modulates the activity of at least one antifibrotic factor in the lungs of the subject.

The present invention also relates to methods of treating severe or rapidly progressing pulmonary fibrosis comprising administering a means for increasing the activity of pulmonary and activation-regulated chemokine (CCL18) in the lungs of the subject, whereby increasing CCL18 activity modulates the activity of at least one antifibrotic factor in the lungs of the subject. The means for increasing the activity of CCL18 include, but are not limited to, those methods described herein, such as transfection, increasing mRNA stability, increasing the biological half-life of a peptide and the like.

The present invention also relates to methods of screening a compound that may alter the progression of severe or rapidly progressing pulmonary fibrosis. The screening methods comprise administering an injury-inducing agent to a control and test population of cells, wherein the injury-inducing agent is known to produce severe or rapidly progressing pulmonary fibrosis. A test procedure is also administered to the test population of injured cells, wherein the test procedure is known or suspected of being able to increase the activity of pulmonary and activation-regulated chemokine (CCL18) in cell populations. Data is observed or gathered wherein the data comprises test level activities of at least one antifibrotic factor in or from the test population of cells. The test data are compared to control data wherein the control data comprises standard activity level of the at least one antifibrotic factor are established in the control population of injured cells. A difference in test activity levels and standard activity levels indicates that the test procedure may be capable of altering the progression of severe or rapidly progressing pulmonary fibrosis.

In one embodiment, the methods of the present invention increase CCL18 expression by gene delivery into tar-et cells.

In one embodiment, the target cells of the present invention are pulmonary cells.

In one embodiment of the methods of the present invention, the gene delivery comprises the use of a viral vector. The viral vector may comprise a replication-deficient recombinant adenoviral vector or replication-deficient recombinant adeno-associated viral vector.

In another embodiment of the methods of the present invention, the gene delivery comprises the use of a non-viral vector.

In one embodiment, the methods of the present invention increase CCL18 expression with viral or non-viral CCL18 gene delivery into target cells. In other embodiments, the methods of the present invention increase CCL18 expression gene delivery comprising magnetofection, cationic lipid-based delivery, electroporation, and a combination thereof.

In one embodiment, the antifibrotic factors include, but are not limited to matrix metalloproteinase-2 (MMP2), matrix metalloproteinase-9 (MMP9), tumor necrosis factor alpha (TNF-α), interleukin-8 (IL-8), interleukin-1 (IL-1), T cells, B cells, natural killer (NK) cells, interferon gamma (IFN-γ), interferon alpha (IFN-α), and combinations thereof.

In another embodiment, increasing CCL18 expression of the present invention modulates the expression of at least one antifibrotic factor selected from the group consisting of MMP2, MMP9, TNF-α, IL-8, IL-1, T cells, B cells, natural killer (NK) cells, IFN-γ, IFN-α, and a combination thereof.

In one embodiment of the methods of the present invention, the severe and/or rapidly progressing fibrosis is mediated by at least one immune cell selected from the group consisting of monocytes, macrophages, lymphocytes, plasma cells, and a combination thereof.

In one embodiment of the methods of the present invention, the immune cells are macrophages.

In another embodiment of the methods of the present invention, the immune cells are lymphocytes.

In one embodiment of the methods of the present invention, the macrophages produce cytokines selected from the group consisting of TNF-α, IL-8, IL-1, and a combination thereof.

In another embodiment of the methods of the present invention, the lymphocytes are selected from the group consisting of T cells, B cells, natural killer (NK) cells, and a combination thereof.

In one embodiment of the methods of the present invention, the lymphocytes are T cells.

In another embodiment of the methods of the present invention, the T cells produce IFN-γ.

In one embodiment of the methods of the present invention, the severe and/or rapidly progressing fibrosis is pulmonary fibrosis.

In one embodiment of the methods of the present invention, the severe and/or rapidly progressing fibrosis is associated with a condition selected from the group consisting of scleroderma lung disease, saracoidosis, Wegener's granulomatosis, infections, asbestosis, ionizing radiation exposure, lupus, rheumatoid arthritis, hypersensitivity pneumonitis, nonspecific interstitial pneumonitis, Hamman-Rich Syndrome, diffuse fibrosing alveolitis, idiopathic pulmonary fibrosis, and combinations thereof.

In one embodiment of the methods of the present invention, the severe and/or rapidly progressing fibrosis is further mediated by metalloproteinases including, but not limited to MMP2, MMP9, or a combination thereof.

In one embodiment, the severe or progressing pulmonary fibrosis is associated with tissue injury.

In another embodiment of the methods of the present invention, the tissue injury is caused by an injury-inducing agent selected from the group consisting of anticonvulsant drug, antipsychotic drug, antidepressant drug, anti-inflammatory drug, anti metabolic drug, antimicrobial drug, biologic response modifiers, cardiovascular drug, chemotherapeutic drug, immunosuppressive drug, and combinations thereof.

In one embodiment of the methods of the present invention, the antimicrobial drug is selected from the group consisting of nitrofurantoin, sulfasalazine, tetracycline, minocycline, sulfonamides, parpa-aminosalicyclic acid, ethambutol, ampicillin, cephalosporin, and combinations thereof.

In one embodiment of the methods of the present invention, the cardiovascular drug is selected from the group consisting of amiodarone, angiotensin-converting enzyme (ACE) inhibitor, and combinations thereof.

In one embodiment of the methods of the present invention, the chemotherapeutic drug is selected from the group consisting of bleomycin, mitomycin-C, busulfan, cyclophosphamide, nitrosourea, procarbazine, melphalan, paclitaxel, and combinations thereof.

### BRIEF DESCRIPTION OF THE DRAWINGS

FIG. 1 depicts hematoxylin and eosin (H&E) staining of lung sections after intratracheal instillation of AdV-CCL18 (A,B), bleomycin (C,D), or their combination (E,F). Instillation of AdV-NULL caused minimal infiltration of inflammatory cells on days 3-7 that completely resolved by day 14. Instillation of AdV-CCL18 and subsequent CCL18 overexpression manifested in peribronchial and perivascular lymphocytic infiltration around small (A) and large (B) bronchi and vessels (arrows), and minimal interstitial lymphocytic infiltration (arrowheads). Instillation of bleomycin resulted in the characteristic diffuse insterstitial alveolitis accompanied by distortion of alveolar architecture (C,D). The combined effect of CCL18 overexpression and bleomycin injury manifested in large areas of distorted alveolar architecture and more pronounced infiltration throughout the lung (E,F), and more severe interstitial lymphocytic infiltration remotely from bronchi and vessels (arrows). Immunohistochemical staining for CD4+ cells (G-I) confirmed the peribronchial and perivascular nature of the infiltration in the CCL18 overexpression model (G), more scattered presence of T cells in the lung parenchyma in the bleomycin injury model (H), and the combined lymphocytic accumulation pattern (adjacent to anatomical structures plus interstitial) upon combined CCL18 overexpression and bleomycin injury to the lungs (I).

FIG. 2 depicts absolute (A) and relative (B) BAL cell count, and total protein concentration in lung homogenates. A. The majority of BAL cells were represented by macrophages and lymphocytes in all groups, with additive effects of CCL18 overexpression and bleomycin on total cell and lymphocyte counts (BLM 0.03 U/mouse shown). B: Stacked column plot showing relative macrophage (top, open bars) and lymphocyte (bottom, shaded bars) content in bronchoalveolar lavage, mean percent±SD of total BAL cells (the averaging procedure may lead to a combine cell counts slightly exceeding 100%). Other cell types were represented by neutrophils and epithelial cells and did not jointly exceed 3% in any of the groups; there was not difference in the neutrophil content between these groups (p>0.05). As previously reported (11), mice instilled with either PBS or AdVNULL (Ctrl) showed no difference (p>0.05). The second instillation was with either PBS or BLM as shown. The differences between Ctrl and CCL18 are significant in all cases (p<0.05, Student's t-test, three to eight animals per group, repeated on three different occasions with consistent results). These data suggest that CCL18 overexpression and bleomycin injury have additive effect on lymphocytic accumulation in the lungs. C: Levels of total protein in lung homogenates in the combined CCL18 overexpression and bleomycin injury group exceeded those in any other group (p<0.05), further suggesting that these two factors facilitate pulmonary inflammation in the additive fashion.

FIG. 3 depicts total hydroxyproline per left lung (Hyp) as a surrogate measure of collagen content. A: 4 Mean Hyp, tag±SD, three to eight animals per group The second instillation was with either PBS or BLM as shown. Notice that in the absence of bleomycin, CCL18 overexpression stimulated collagen accumulation (p<0.05). In contrast, CCL18 overexpression partially neutralized the effect of bleomycin on collagen accumulation (p<0.05 for both doses of bleomycin). The expected additive effect of CCL18 overexpression and bleomycin injury on collagen accumulation is shown with the unfilled bars/dashed lines. B: Average Hyp level per lung in six independent experiments as described in A for BLM 0.03 U; standard deviations were similar to those shown in A. Each point indicates average Hyp, presented as percent of Hyp value in corresponding PBS-treated controls, three to eight animals per group. The connecting lines represent six independent experiments performed on separate occasions.

FIG. 4 depicts matrix metalloproteinases (MMP) in the lungs of mice overexpressing CCL18 and/or treated with bleomycin. A,B: ELISA of lung homogenates for total (pro- and active) MMP2 (Panel A) and pro-MMP9 (Panel B). Data are shown as mean pg/pg total protein±SD, eight to twelve animals per group, repeated on two different occasions with similar results. Overexpression of CCL18 and injury with bleomycin act additively on accumulation of matrix metalloproteinases. In Panel A, the differences between CCL18-expressing and non-expressing animals were significant (p<0.05) in each bleomycin dose group. Similar tendencies were observed in Panel B although the differences between CCL18-expressing and non-expressing animals did not reach significance (p>0.05) within each bleomycin dose group, due to low concentration of pro-MMP9 and low signal-to-noise ratio. C: Immunohistochemistry for MMP9 (pro- and total), ×200 magnification, showing additive effect of CCL18 overexpression and bleomycin exposure on accumulation of MMP9-producing cells in the lung (brown staining). No staining was detected with isotype control antibody (not shown) D: Zymogram of lung homogenates. Sample loading was normalized to total protein (Bio-Rad assays). Molecular weight markers, kDa, are indicated on the right. Expected locations of pro-MMP9 (92 kDa), active MMP9 (82 kDa), pro-MMP2 (72 kDa), and active MMP2 (62 kDa) are indicated with arrows. Repeated on three separate occasions in different groups of animals, with similar results.

FIG. 5 depicts ELISA of lung homogenates for IFN-γ(A), TNF-α(B), MCP-1 (C), and active TGF-β (D), pg/ml, mean±SD. Data averaged from three to eight animals per group in (A,B,C) and eight to twelve animals per group in (D). The increases in IFN-γ (A), TNF-α (B), and MCP-1 (C) in the CCL18+BLM group were significant (p<0.05) in comparison with any other group and appeared additive of the effects of CCL18 alone and BLM alone. In (D), the level of active TGF-β in the CCL18+BLM group was different (p<0.05, Student's t-test) from any other group, but not additive of the effects of CCL18 alone and BLM alone. In these experiments, 0.03 U BLM was utilized.

FIG. 6 depicts changes in the total levels of hydroxyproline, fold increase versus control, in the lung of mice overexpressing CCL18 and challenged with high dose of bleomycin, upon treatment with an MMP inhibitor GM6001 or neutralizing anti-MMP9 antibody. Both treatments significantly abrogated accumulation of hydroxyproline and cytokine levels where indicated with asterisks (p<0.05, Student's t-test, three to eight animals per group). Treatment with GM6001 in the group of mice instilled with 0.03 U of bleomycin alone did not attenuate the levels of hydroxyproline (207.3±18.8 pg/lung vs 222.7±15.6 pg/lung, non-treated vs treated groups, respectively, p>0.05).

FIG. 7 is shows the antifibrotic effect produced by increasing CCL18 expression in the lungs, and the advantages of increasing CCL18 to prevent or treat pulmonary fibrosis. The claimed method of increasing CCL18 is compared to the method of injecting recombinant antifibrotic cytokines to reduce pulmonary fibrosis. The injection of recombinant anti fibrotic cytokines failed to reduce pulmonary fibrosis because the half-life of the injected recombinant cytokine is short, and its bioavailability in the pulmonary tissues is minimal. In contrast, the increase in CCL18 expression produced by the present invention causes an unexpected enhancement in the observed levels of antifibrotic factors, e.g., by T lymphocytic infiltration and/or production of IFN-γ and TNF-α, into the lungs. The attracted T cells become an important local source of antifibrotic cytokines, and is advantageous because the infiltration of T cells allow for: 1) the continuous production of antifibrotic cytokines that, in comparison to the method of injecting cytokines, is independent of the amount of application and half-life of the cytokine, and 2) local, tissue specific production of antifibrotic factors which, in comparison to the method of injecting cytokines, ensure for substantial bioavailability of the protective antifibrotic cytokines in the tissues. Because the claimed method provides for a local source of T cells, the present invention also circumvents the systemic side effects and problems associated with crossing the endothelial barrier that is encountered by the injection of cytokines.

### DETAILED DESCRIPTION OF THE INVENTION

The present invention relates to methods of treating severe or rapidly progressing pulmonary fibrosis in a subject in need of a treatment thereof. The methods comprise increasing the activity of pulmonary and activation-regulated chemokine (CCL18) in the lungs of the subject, whereby increasing CCL18 activity modulates the activity of at least one antifibrotic factor in the lungs of the subject.

CCL18 is a profibrotic “CC chemokine” that is chemotactic for T cells that is constitutively expressed in the lungs. CCL18 has a high amino acid sequence identity with macrophage inflammatory protein-1 alpha (MIP-1a), but does not bind to the MIP-1a receptors CCR5 and CCR1. Monocyte chemotactic protein-1 (MCP-1) is the only other known CC chemokine capable of increasing collagen production in fibroblasts. CCL18 promotes fibrosis and is expressed at high levels in the lungs, particularly by activated lung macrophages, although other tissue macrophages and dendritic cells may secrete CCL18.

In addition to its profibrotic activity, CCL18 attracts naive and activated CD4+ and CD8+T cells. Fibrosis was observed in animals infected with a replication-deficient adenovirus harboring the CCL18 gene. The levels of CCL18 produced in adenoviral models may, however, be sufficient to attract T-cells, which may, in turn, be contributing to collagen accumulation and fibrosis observed in the adenovirus-infected animals.

CCL18 is a cytokine that is differentially regulated in classically and alternatively activated macrophages. For example, interferon-7 inhibits CCL18 production in activated macrophages, whereas interleukin 4 (IL-4), IL-13, and IL-10 induce CCL18 production. Furthermore, the development of pulmonary fibrosis is generally associated with predominant expression of type 2 cytokines in the lungs, thus type 2 cytokines not only promote lung fibrosis by acting directly on lung, fibroblasts, but also indirectly through alternative pathway to increase production of CCL18.

CCL18 increases the phosphorylation of extracellular signal-regulated kinase (ERK), a kinase involved in a variety of second messenger cell signaling cascades, in a time-dependent manner. In addition, pharmacological inhibition of ERK blocked the CCL18-induced stimulation of collagen production in fibroblast. Accordingly, CCL18 directly stimulates collagen production in lung and dermal fibroblasts by activating intracellular signaling through the ERK pathway.

CCL18 directly stimulates type I collagen production in at least lung and dermal fibroblasts. This increase in collagen mRNA indicates that either an increase in gene transcription or an increase in mRNA stability may be responsible for the increased collagen production in response to CCL18. It is possible that CCL18 may also affect the intracellular pools of free proline, thus accounting for, at least in part, CCL18's stimulation of collagen production in fibroblasts.

For the purposes of the present invention, “CCL18” includes full length CCL18 protein, as well as functional fragments thereof. Using standard assays to measure for typical CCL18 activity, one of skill in the art could assay for functional fragments of CCL18. The fragments need not be as active or effective as full length, provided that the CCL18 fragments are derived from the full-length CCL18 and that the fragments have at least one activity associated with the full-length CCL18 protein.

Activated alveolar macrophages are an abundant source of CCL18, and lung macrophages are actively involved in lung inflammation involved in pulmonary fibrosis. Studies have shown that CCL18 is in elevated concentrations in BAL fluids taken from patients with scleroderma lung disease.

The present inventors have recently reported that mice infected with a replication-deficient adenovirus encoding CCL18 but not with a similar control virus develop selective T lymphocytic infiltration of the lungs, as well as moderate transient T cell-dependent collagen accumulation (11). Phenotypic characterization of the infiltrating cells in comparison with normally present pulmonary T cells revealed minimal, if any, activation, including lack of elevated expression of several profibrotic factors (1). However, the lymphocytic infiltration coincided with the sites of accumulation of active TGF-131 and collagen (11), suggesting that the infiltrating T cells directly contributed to the profibrotic effect of CCL18. In support of this notion, systemic depletion of T cells completely abrogated lymphocytic infiltration and collagen accumulation in CCL18-overexpressing mice (11).

In patients with lung fibrosis, increases in pulmonary levels of CCL18 occur in association with lung inflammation and fibrosis (1-7). The lung inflammation is characterized by an influx of lymphocytes, collagen accumulation, and increase in proinflammatory factors such as chemokines, macrophage inflammatory protein 4 (MIP-4), alternative macrophage activation-associated CC chemokine 1 (AMAC-1), dendritic cell-derived chemokine 1 (DCCK1), and small secreted cytokine A 18 (SCYA-18).

As used herein, the “activity of CCL18” is used to mean the expression of CCL18 or the actions or effects of CCL18. Thus, increasing the activity of CCL18 within a cell would, for the purposes of the present invention, include increasing the expression of CCL18. Similarly, increasing the stability of mRNA that codes for CCL18 would also constitute increasing the activity of CCL18. Of course, increasing the activity of CCL18 would also include administering the CCL18 peptide directly or indirectly to the target cells. In one embodiment, the methods of increasing the activity of CCL18 include, but are not limited to, increasing the expression of CCL18 in a cell or population of cells by delivering one or more non-native nucleic acids to a target cell that codes for CCL18 or a peptide with CCL18 activity. The nucleic acid delivery into target cell can be accomplished using standard transfection techniques, which include, but are not limited to, magnetofection, cationic lipid-based delivery, or electroporation. The nucleic acid delivery may be via a viral or non-viral vector mediated delivery into the target cells. As used herein, a “non-native” nucleic acid is intended to mean a nucleic acid that the target cell does not normally contain. Thus, a “non-native nucleic acid” includes, but is not limited to, an extra copy of a nucleic acid that codes for CCL18, even if the nucleic acid is wild-type to the target cell. Of course, a “non-native nucleic acid” also includes, but is not limited to, nucleic acids that are heterologous to the target cell.

The nucleic acid constructs can be any construct capable of delivering non-native nucleic acids to target cells, and the invention is not limited to or dependent upon a particular type of construct for delivery of the nucleic acid encoding for CCL18. Examples of vectors and plasmids are abundant in the art and commercially available. In one embodiment, viral vector are used to increase the expression of CCL18 and include, but are not limited to, replication-deficient recombinant adenoviral vectors and replication-deficient recombinant adeno-associated viral vectors.

In accordance with the present invention, the “target cell” may include, but is not limited to, pulmonary cells, epithelial cells, cerebral cells, breast cells, myocardial cells, musculoskeletal cells, liver cells, neuronal cells, vascular cells, vein cells, skin cells, pancreas cells, spleen cells, gall bladder cells, kidney cells, urogenital cells, ocular cells, or other cells susceptible to fibrosis, provided that the cell produces or causes a response to the increased activity of CCL18.

In accordance with the present invention, a “CCL18-responsive cell” is a cell in which an increase in expressed CCL18 can trigger a biological response either in vitro or in vivo. Such cells may include, but are not limited to, T cells, B cells, dendritic cell chemokine (DC-CK1), hematopoietic progenitor cells, fibroblasts, monocytes, macrophages, or a combination thereof. For instance, CCL18 may trigger T cell production of cytokines such as IFN-γ, or activate monocytes or macrophages to produce cytokines such as T helper cell type 2 (Th2)-related cytokines (e.g., IL-4, IL-10, or IL-13), or glucocorticoids (GC).

The present invention relates to methods of treating severe or rapidly progressing pulmonary fibrosis. As used herein, the term “treatment” is used to indicate a procedure which is designed ameliorate one or more causes, symptoms, or untoward effects of an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. Thus, a treatment may include treating a subject to attenuate symptoms such as, but not limited to, discomfort, pain, shortness of breath (particularly with exertion), chronic dry, hacking, cough, fatigue and weakness, discomfort in the chest, loss of appetite and rapid weight loss, in a subject, or may include removing or decreasing the severity of the root cause of the abnormal condition in the subject. Treatment also includes treating after-arising symptoms that are related to the initiation pulmonary fibrosis.

As used herein, the term “subject” is used interchangeably with the term “patient,” and is used to mean an animal, in particular a mammal, and even more particularly a non-human or human primate.

The term “fibrosis” is used herein as it is in the medical arts and refers to the formation or development of fibrous connective tissue in an organ or tissue due to collagen and/or other connective tissue accumulation. A molecule that promotes fibrosis is one that directly or indirectly contributes to the accumulation of collagenous and/or other connective tissue.

The term “severe and/or rapidly progressing fibrosis” is used to refer to significant overgrowth, scarring or hardening that occurs throughout a particular organ or tissue, e.g., the lungs, and indicates an abnormal condition in a subject that is marked by excessive accumulation of collagenous and/or other connective tissue in comparison to a normal condition in which the fibrous tissue is a normal constituent of an organ or tissue. The abnormal condition causes the formation or development of fibrous connective tissue from excessive collagen and/or other connective tissue accumulation in an organ or tissue as a result of a reactive process, in contrast to a formation of fibrous tissue that is a normal constituent of an organ or tissue. Examples of pathologic and excessive fibrotic accumulation include, but are not limited to, pulmonary fibrosis, benign prostate hypertrophy, fibrocystic breast disease, uterine fibroids, ovarian cysts, endometriosis, coronary infarcts, cerebral infarcts, myocardial fibrosis, musculoskeletal fibrosis, post-surgical adhesions, liver fibrosis, cirrhosis, real fibrotic disease, or fibrotic vascular disease, e.g., atherosclerosis, varix, or varicose veins, scleroderma, Alzheimer's disease, diabetic retinopathy and glaucoma.

Severe and/or rapidly progressing fibrosis may be determined based on an increase in collagen accumulation over normal individuals, as clinically judged by lower pulmonary functions measures, including, but not limited to, diffusing capacity for carbon monoxide (DLCO) and forced vital capacity (FVC). The increase in collagen and/or other connective tissue accumulation over normal individuals may be evaluated according to known methods for measuring tissue collagen accumulation and using histological analyses evaluating changes in pulmonary architecture and collagen-specific staining, including, but not limited to, trichrome staining. The extent of collagen and/or other connective tissue accumulation in severe and/or rapidly progressing fibrosis over normal individuals may, in turn, be determined by using standard methods known in the art to measure the molecular and cellular changes in cells and tissues that are related to fibrogenesis. Severe and/or rapidly progressing fibrosis may be determined based on, for example, a combination of clinical changes (including, but not limited to, dyspnea, cough, bibasilar crackles), pulmonary function changes (including, but not limited to, worse DLCO and FVC values), and radiographic and histological changes consistent with interstitial pneumonia. In parallel, the diagnosis may be confirmed by measuring the levels of biomarkers in the lungs that characterize the pathogenesis of fibrosis including, but not limited to, profibrotic growth factors, reactive oxygen species (ROS), cell signaling factors, and proinflammatory cytokines. Profibrotic growth factors include, but are not limited to, transforming growth factor-beta (TGF-β), connective tissue growth factor (CTGF), or a combination thereof. For example, TGF-β is believed to be a key mediator of tissue fibrosis as a consequence of extracellular matrix (ECM) accumulation in pathologic states such as scleroderma. TGF-β is known to induce the expression of ECM proteins in mesenchymal cells, and to stimulate the production of protease inhibitors that prevent enzymatic breakdown of the ECM. CTGF, which is induced by TGF-β, has been reported to mediate stimulatory actions of TGF-β ECM synthesis. Furthermore, severe and/or rapidly progressing fibrosis may be determined using standard methods known in the art to measure levels of antifibrotic factors such as IFN-γ and/or TNF-α.

In one embodiment, the severity of pulmonary fibrosis is evaluated using a standard hydroxyproline assay. It is well-known that hydroxyproline is incorporated into collagen, thus levels of hydroxyproline can be directly correlated to levels of collagen from a sample. Furthermore, it is also known that fibrosis may not be uniform throughout an affected organ, and that the fibrosis may be “patchy.” Thus, for the purposes of the present invention, “moderate fibrosis” is intended to mean an organ or tissue, for example the lung, where at least a portion of the organ or tissue has greater than a 1 to about a 1.5 fold increase in the levels of hydroxyproline over levels of hydroxyproline in the organ or tissue in subjects that do not have fibrosis. “Severe firbrosis” is intended to mean an organ or tissue, for example the lung, where at least a portion of the organ or tissue has greater than about a 1.5 increase in the levels of hydroxyproline over levels of hydroxyproline in the organ or tissue in subjects that do not have fibrosis. It is well-known to those of ordinary skill in the art that other means of measuring and determining fibrosis are known in the art with such other means of measuring and determining fibrosis representing a further embodiment of the present invention.

As used herein, “about” refers to a numeric value, including, for example, whole numbers, fractions, and percentages, whether or not explicitly indicated. The term “about” generally refers to a range of numerical values (e.g., +/−5-10% of the recited value) that one would consider equivalent to the recited value (e.g., having the same function or result). In some instances, the term “about” may include numerical values that are rounded to the nearest significant figure.

In one embodiment of the present invention, the fibrosis that is treated or prevented from progressing by the methods described herein is pulmonary fibrosis. In more particular embodiments, the pulmonary fibrosis is a symptom of a condition including, but not limited to, scleroderma lung disease, saracoidosis, Wegener's granulomatosis, infections, asbestosis, ionizing radiation exposure, lupus, rheumatoid arthritis, hypersensitivity pneumonitis, nonspecific interstitial pneumonitis, Hamman-Rich Syndrome, diffuse fibrosing alveolitis, idiopathic pulmonary fibrosis, or a combination thereof.

The severe or rapidly progressing fibrosis ay also be associated with tissue injury. As used herein, the term “tissue injury” is used to mean damage or harm caused to the structure or function of a tissue by an agent that may be physical or chemical. The injury induced by an agent to a target cell in the present invention may cause a variety of symptoms, in particular, fibrosis and inflammation. The agent of the present invention may also cause injury by exacerbating an underlying disease in a predisposed subject, or cause the disease.

In one embodiment, the injury could be cellular injury. The injury may be mediated by at least one immune cell selected from monocytes, macrophages, lymphocytes, plasma cells, and a combination thereof. In particular, the injury by an injury-inducing agent of the present invention is to a pulmonary cell resulting in pulmonary injury that causes pulmonary fibrosis and/or pulmonary inflammation.

As used herein, “cellular injury” to a target cell may be reversible or irreversible. Cellular injury includes, but is not limited to, cellular swelling (cellular hypertrophy), cellular atrophy (cell shrinkage), fatty change (cells fail to metabolize fatty acids and accumulate lipids), or a combination thereof. Additional examples of cellular injury include, but are not limited to, changes in the density of the mitochondrial matrix, cell membrane disruption, nuclear shrinkage (pyknosis), nuclear dissolution (karyolysis), nuclear break up (karyorrhexis), lysosome rupture, apoptosis, cellular necrosis, cellular hyperplasia (an increase in the number of cells which may have increased cellular volume caused by physiological stress or pathological stimuli), or a combination thereof.

The structure and function of a cell are interdependent. An injurious agent may target a particular aspect of a cell structure or function and lead to cellular injury. Mechanisms of cellular injury include, but are not limited to, cell membrane damage (e.g., complement-mediated lysis via the membrane attack complex, bacterial toxins, free radicals), mitochondrial damage leading to inadequate aerobic respiration (e.g., hypoxia, cyanosis), ribosomal damage leading to altered protein synthesis (e.g., alcohol, antibiotics), increased production of reactive oxygen or nitrogen species, and nuclear damage (e.g., viruses, radiation, free radicals). Potential causes of cellular injury include, but are not limited to, hypoxia, immunological, infection by microorganisms, genetic, physical, and chemical, such a drug-related fibrosis.

The pulmonary injury induced by at least one injury-inducing agent includes, but is not limited to, pulmonary fibrosis and pulmonary inflammation. The pulmonary injury may be mediated by at least one immune cell selected from the group consisting of monocytes, macrophages, lymphocytes, plasma cells, and a combination thereof.

The agent that may be used to induce pulmonary injury includes, but is not limited to, anticonvulsant drug, anti-inflammatory drug, antimetabolic drug, antimicrobial drug, biologic response modifiers, cardiovascular drug, chemotherapeutic drug, immunosuppressive drug, illicit drug, or a combination thereof.

The anticonvulsant drug may include, but is not limited to, carbamazepine, chlordiazepoxide, fluoxetine, phenothiazines, phenytoin, trazodone, tricyclics, or a combination thereof.

The anti-inflammatory drug may include, but is not limited to, aspirin, gold, methotrexate, penicillamine, or a combination thereof.

The antimetabolic drug may include, but is not limited to, azathioprine, cytarabine, fludarabine, gemcitabine, 6-mercaptopurine, methotrexate, or a combination thereof.

The antimicrobial drug may include, but is not limited to, nitrofurantoin, sulfasalazine, tetracycline, minocycline, sulfonamides, parpa-aminosalicyclic acid, ethambutol, ampicillin, cephalosporin, or a combination thereof.

The biologic response modifiers may include, but are not limited to, granulocyte-macrophage colony-stimulating factor, interferon, interleukin-2, tumor necrosis factor, or a combination thereof.

The cardiovascular drug may include, but is not limited to, amiodarone, angiotensin-converting enzyme (ACE) inhibitor, or a combination thereof.

The chemotherapeutic drug may include, but is not limited to, bleomycin, mitomycin-C, busulfan, cyclophosphamide, nitrosourea, procarbazine, melphalan, paclitaxel, or a combination thereof.

The immunosuppressive drug may include, but is not limited to, cyclosporine, corticosteroids, azathioprine, cyclophosphamide, or a combination thereof.

The illicit drug may include, but is not limited to, cocaine, heroin, methadone, methylphenidate, narcotic, sedative, or a combination thereof.

The treatment methods of the present invention are used to modulate the activity of at least one anti-fibrotic factor in the lungs of a subject. As used herein, the term “modulate” means to vary, alter, or change. For instance, the injury-inducing agent of the present invention modulates fibrosis by increasing antifibrotic or decreasing profibrotic factors that play a role in the formation or development of excess fibrous connective tissue in an organ or tissue.

As used herein, the term “antifibrotic” means regression of fibrosis. Alternatively, the term “profibrotic” means progression of fibrosis. “Regression” as used herein means a lessening of symptoms or reduction of the accumulation of collagenous and/or other connective tissue or the reduction of the total amount of excessive collagen in a particular tissue, relating to the fibrotic condition before employing the methods of the present invention without complete disappearance of the symptoms. “Progression” as used herein means advancement or progressing of symptoms.

The antifibrotic factors include, but are not limited to, metalloproteinases, interleukins, interferons, cytokines, chemokines, chemotactic molecules, macrophages, lymphocytes, or a combination thereof. In particular, the antifibrotic factors include but are not limited to matrix metalloproteinase-2 (MMP2), MMP9, tumor necrosis factor alpha (TNF-α), T cells, interferon gamma (IFN-γ), and combinations thereof. Other antifibrotic factors are well-known in the art and need not be repeated herein.

In one embodiment, the methods comprise administering to a subject an effective amount of a compound or molecule that increases the activity of CCL18. As used herein, the term “effective amount” means an amount that is sufficient to achieve the stated or desired effect, and can be a simple matter of titration. An effective amount of CCL18 used in the methods of the present invention is an amount sufficient to increase the activity, e.g., concentrations, of CCL18 in a target cell in comparison to an untreated (control) cell.

An effective amount of the drug, as the term is used herein, refers to that amount of drug which is effective therapeutically in the desired treatment. In accordance with the present invention, the effective amount of an agent used in the present methods is one that elicits any one or all of the effects often associated with the in vivo biological activity of the agent. In addition, the effective amount of an agent in the present methods may also include one that elicits an in vitro biological effect by agent.

For instance, in accordance with the present invention, an effective amount of CCL18 may be below 1 ng/ml to initiate attraction of T cells to the lungs. Alternatively, at the concentrations above 1 ng/ml, up to 300 ng/ml or 1000 ng/ml or more, an effective amount used in the methods is likely to act not only to attract T cells to the lungs, but also directly to affect pulmonary fibroblasts. These concentrations refer to the average concentrations measured by standard techniques such as ELISA or Western blotting in lung homogenates or alternatively, to local concentrations in the immediate vicinity of cells such as T cells or fibroblasts.

A medicament useful for the methods of treating, preventing or preventing the progression of fibrosis may be prepared by standard pharmaceutical techniques known in the art, depending upon the mode of administration and the particular disease to be treated. The medicament will usually be supplied as part of a sterile, pharmaceutical composition which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

Dosages of the substance of the present invention can vary between wide limits, depending upon the disease or disorder to be treated, the age and condition of the individual to be treated, etc. and a physician will ultimately determine appropriate dosages to be used.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions. In certain situations, delayed release preparations may be advantageous and compositions which can deliver, for example, AET or a derivative thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

The present invention also relates to methods of screening a compound that may alter the progression of severe or rapidly progressing pulmonary fibrosis. The screening methods comprise administering an injury-inducing agent to a control and test population of cells, wherein the injury-inducing agent is known to produce severe or rapidly progressing pulmonary fibrosis. A test procedure is also administered to the test population of injured cells. In one specific embodiment, the test procedure is known or suspected of being able to increase the activity of pulmonary and activation-regulated chemokine (CCL18) in cell populations. In response to the test procedure, data is observed or gathered wherein the data comprises test level activities of CCL18 activity. In one specific embodiment, levels of CCL18 are determined using the activity of at least one antifibrotic factor in or from the test population of cells. The test data are compared to control data, wherein the control data comprises standard activity levels of CCL18 and/or the activity levels of the at least one antifibrotic factors that are established in the control population of injured cells. A difference in test activity levels and standard activity levels indicates that the test procedure may be capable of altering the progression of severe or rapidly progressing pulmonary fibrosis.

The test procedures that are used in the screening methods can be any procedure that is known or suspected of being able to increase the activity of CCL18 in a target cell population. Such test procedures include, but are not limited to, increasing expression or biological half-life of proteins that stimulate or are believed to stimulate CCL18 production. Other test procedures include, but are not limited to, administering compounds or compositions to the test cells that can increase expression, increase the stability of mRNA or increase the half-life of the CCL18 peptide. Once a relationship is established between the test procedure and CCL18, regardless of the setting, the test procedure is a candidate for use in the present invention.

Data is collected or gathered towards at least one antifibrotic factor as described herein and the test data are compared to a standard. The standard may be any set point, provided the set point can be compared to the test data. For example, the standard may be the levels concentration or activity of a particular antifirotic factor in a normal cell or tissue, such as a lung. Alternatively, the standard may be the levels, concentration or activity of a particular antifirotic factor in a slightly fibrotic cell culture or tissue. Or the standard may be the levels/concentration or activity of a particular antifirotic factor in a severely fibrotic cell or tissue. The setting of the screening methods may be any setting where data may be gathered such as, but not limited to, a cell culture setting, a tissue mount or a whole animal, etc. The standard activity levels may or may not be known prior to the commencement of the screening methods, and may established at any time.

### INDUSTRIAL APPLICABILITY

The claimed invention provides unexpected and superior results that are advantageous over previous therapies and methods of treating and/or preventing fibrosis, e.g., pulmonary fibrosis. The claimed methods allow for: 1) continuous infiltration of T cell infiltration and infiltration of antifibrotic factors (e.g., IFN-γ), 2) bioavailability of antifibrotic factors in the lungs due to localization of antifibrotic factors in the lung tissues, 3) minimal side effects related to systemic administration of antifibrotic factors. In embodiments where the activity of CCL18 is not increased systemically, problems related to crossing the endothelial barrier are circumvented.

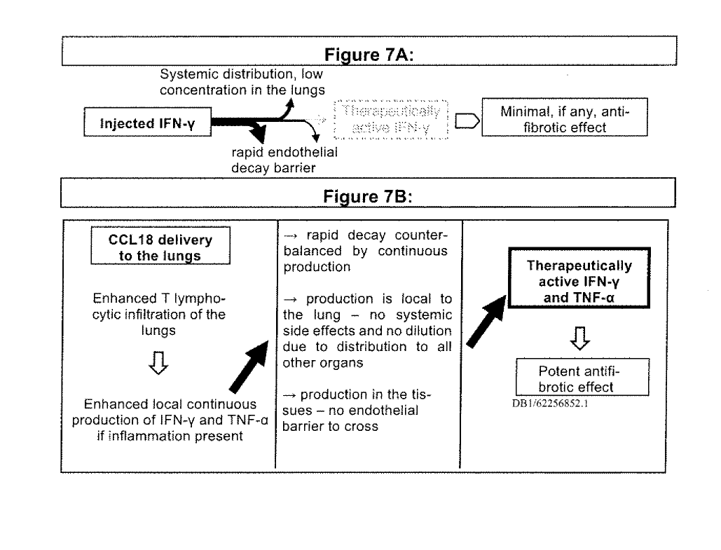

For instance, in the past, antifibrotic cytokines (e.g. IFN-γ) have been therapeutically administered by systemic injection, but failed to reduce pulmonary fibrosis because the half-life of the injected recombinant cytokine is short, and its local bioavailability in mammalian (e.g., human) tissues (e.g., the lungs) is minimal, likely due to the endothelial barrier. In contrast, the present invention allows for an elevated and continuous T-cell lymphocytic infiltration as a result of the increased CCL18 activity, e.g., expression, in the inflammatory milieu of the lung, i.e., increased CCL18 expression causes a continuous elevation of antifibrotic cytokines such as IFN-γ and TNF-α. Because increasing the activity of CCL18 by the claimed method may not involve systemic injections of antifibrotic cytokines, the present invention may circumvent the systemic side effects and minimal bioavailability associated with injecting cytokines (e.g., crossing the endothelial barrier). Thus, the T cells that infiltrate because of the delivery of CCL18 become an important local source of antifibrotic cytokines, and importantly, such source is 1) continuous and independent of injections and the half-life of the cytokine, 2) local and tissue-specific, and 3) ensures substantial bioavailability of protective cytokines in the tissues (because there is no need for the cytokines to cross the endothelial barrier). FIGS. 7A and 7B summarizes the advantageous and unexpected antifibrotic effects of increasing CCL18 expression by the claimed method.

### EXAMPLES

### Example 1

### Experimental Animal Models

Ten- to twelve-week-old C57BL/6 female mice weighing 18-21 g were purchased from The Jackson Laboratory (Bar Harbor, Me.) and maintained in sterile microisolator cages with sterile rodent feed and acidified water. Daily maintenance of mice was performed in the Baltimore Va. Medical Center Research Animal Facility that is approved by the Association for Assessment and Accreditation of Laboratory Animal Care (AAALAC). The animals were treated in accordance with a research protocol that has been approved by the University of Maryland Institutional Animal Care and Use Committee (IACUC). Mice were weighed daily using a calibrated scale.

Recombinant adenoviral vectors AdV-CCL18 and AdV-NULL were constructed, validated, and used as previously reported (11). Intratracheal instillation of 50 μl of bleomycin solution (Sigma-Aldrich, St. Louis, Mo.) or PBS was performed in a fashion similar to instillation of adenoviruses (11).

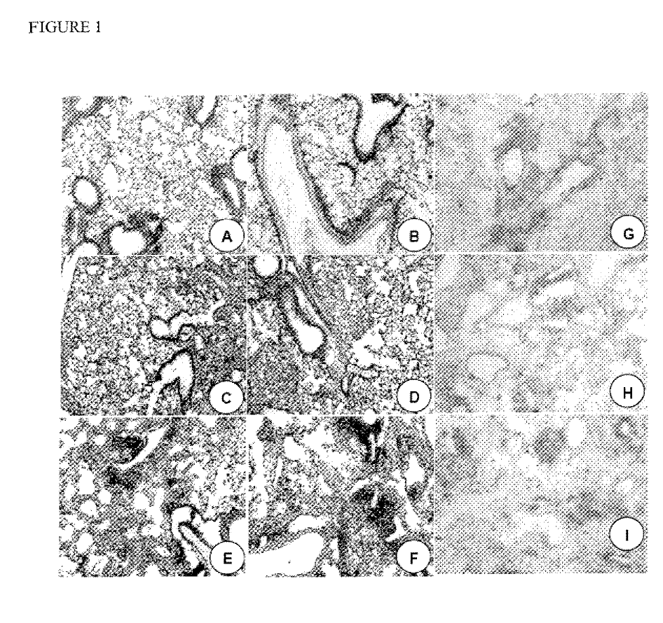

To address the possibility that the effects of CCL18 on collagen production in vivo may depend on the presence and severity of the inflammatory milieu, intratracheal instillation of AdV-CCL18 or, as controls, of AdV-NULL or PBS was performed (day 0). Selected animals were sacrificed to confirm the expected CCL18 expression dynamics as previously reported (see FIG. 1 in ref. 11). At the peak of CCL18 production (day 7), intratracheal instillation of bleomycin was performed to induce lung inflammation and fibrosis; alternatively, PBS was instilled as control.

As a result of these two instillations, four groups of mice were formed, as described below. The control group (Ctrl) was instilled first with AdV-NULL or PBS and then with PBS again. It has been previously reported that mice instilled with AdV-NULL or PBS were not phenotypically or histologically different beyond three days following instillation (11). The CCL18 alone group overexpressed CCL18 as a result of AdV-CCL18 instillation. The second instillation in this group was with PBS. The bleomycin alone (BLM) group received the first instillation of either AdV-NULL or PBS, and the second instillation of either 0.01 U or 0.03 U of bleomycin.

These doses of bleomycin were selected as insufficient to achieve a plateauing effect on lung inflammation and fibrosis, based on the results of preliminary experiments in which titrated doses (0.005 U-0.1 U per mouse) were instilled. Finally, the combined CCL18 overexpression and bleomycin injury group (CCL18+BLM) received AdV-CCL18 (first instillation) and bleomycin (second instillation). On day 21 following the first instillation (day 14 after the second instillation), mice were euthanized by 002 asphyxiation followed by cervical dislocation. ELISA assays of lung homogenates were used to confirm the expected decline of CCL18 levels by day 21 following AdV-CCL18 instillation (11); such decline was independent of the nature of the agent used for the second instillation (bleomycin or PBS).

For depletion of lymphocytes, some of the animals were first injected with antilymphocyte serum (ALS; Accurate, Westbury, N.Y.), on days −4, −2, and 0 relevant to the first intratracheal instillation, and the decrease in the amount of lymphocytes to <5% of the initial levels was confirmed by flow cytometry, as described (11). In some other cases, mice were treated with a broad-spectrum MMP inhibitor GM6001 (Chemicon, Temecula, Calif.) intraperitoneally at 2 mg/mouse daily for the last 5 days before euthanasia; or with anti-MMP9 neutralizing antibody (Calbiochem, San Diego, Calif.) intraperitoneally at 60 fag/mouse on days 14 and 18 after first instillation.

### Example 2

### Histological Examination of the Lungs, Bronchoalveolar Lavage, and Flow Cytometry

Immediately postmortem, the lungs were rapidly dissected free of extraneous tissues and filled with either formalin-free fixative (Anatech, Battle Creek, Mich.) for subsequent hematoxylin and eosin staining or with 1:1 mixture of PBS and TissueTek OCT compound (Sakura, Torrance, Calif.) for subsequent immunohistochemical analyses. Cryostat sections, stainings, controls, and image analyses were performed as described (11).

For BAL, the animals were euthanized, and lung lavage was performed immediately postmortem through an 18-gauge blunt-end needle secured in the trachea as described (11). Differential cell count in BAL samples were performed after staining of cytospin preparations with a Protocol Hema 3 staining set (Fisher, Kalamazoo, Mich.) by at least two technicians who were blinded to the identity of the samples. The flow cytometric analyses of BAL cells were performed after staining with directly labeled antibodies (BD PharMingen, San Diego, Calif.) or corresponding isotype controls as described (11).

### Example 3

### Determination of Hydroxyproline Content ELISA Analyses, and Zymographic Analyses of Lung Homogenates

Pulmonary levels of hydroxyproline were measured as surrogate of total collagen, as described (11). Briefly, the snap-frozen lungs were crushed under liquid nitrogen, thawed in 0.5 ml of PBS containing a protease inhibitor cocktail (Sigma), and further homogenized in a glass homogenizer. The solid tissue was separated by centrifugation; the supernatant was diluted two fold with the ELISA sample buffer, and used for ELISA analyses of total and active TGF-β, IL13, TNF-α, IFN-γ, MCP-1 (CCL2), total (pro- and active) MMP-2, and pro-MMP-9 (all kits purchased from R&D Systems, Minneapolis, Minn.).

Total protein was measured using Bio-Rad assay (Hercules, Calif.). The solid tissue was hydrolyzed in 5N NaOH at 120° C. for 30 minutes in an autoclave. The mixture was then reacted with chloramine T and Ehrlich's reagent to produce a chromophore, which was quantified by spectrophotometry at 550 nm. A second aliquot of the original lung homogenate was used for colorimetric detection and quantification for total protein content using Bio-Rad assay.

For zymographic analyses, lungs were homogenized in 50 mM Iris-NCI buffer containing 1 mM monothioglycerol, and the solid tissue was separated by centrifugation. The supernatants were normalized for total protein and loaded onto Novex® 10% zymogram gels containing 0.1% gelatin (Invitrogen, Carlsbad, Calif.). After electrophoretic separation gels were renatured, developed at 37° C. overnight, and stained with Colloidal Blue stain (Invitrogen) following manufacturer's recommendations.

### Example 4

### In Vitro Chemotaxis Assays

Chemotaxis assays were used as described in (11) to determine whether CCL18 selectively attracts regulatory T cells. Briefly, human T cells purified from PBMC as described in (11) were seeded in triplicates in the upper chamber using Costar Transwell inserts (3-pm pore size; Costar, Cambridge, Mass.) an incubated, with or without rhCCL18 in the lower chamber, at 37° C. for 4 hours. The cells that migrated into the lower chamber, as well as the cells that remained in the upper chamber, were analyzed for expression of cell surface CD4, CD25, and intracellular FoxP3 by flow cytometry. Data are reported as the mean±SD. Differences between groups were evaluated with Student's 2-tailed unequal variance t-test and Mann-Whitney U test P values less than 0.05 were considered statistically significant.

### Example 5

### Pulmonary Responses to Adenoviral Delivery of CCL18 and Instillation of Bleomycin

To evaluate the combined effect of CCL18 overexpression and bleomycin injury on the lungs, each animal in EXAMPLE 1 received two intratracheal instillations. The first instillation of AdVCCL18 or AdV-NULL or PBS, was followed by the second instillation of bleomycin or PBS, as described in EXAMPLE 1.

Mice instilled intratracheally with AdV-CCL18, AdV-NULL, or PBS showed no signs of morbidity such as body weight loss (p>0.05, one-way ANOVA), ruffled fur, dehydration, diarrhea, hunched posture, or decreased motor activity at any time postinfection. Mice instilled with bleomycin showed an expected total body weight loss of maximum 6.2±2.7% following instillation of 0.01 U of bleomycin and maximum 10.1±3.2% following instillation of 0.03 U of bleomycin. This statistically significant (p<0.05 by one-way ANOVA, data not shown) weight loss was observed on days 5-20 following the second intratracheal instillation of bleomycin but not PBS, independent of the nature of the agent used for the first instillation (AdV-CCL18, AdV-NULL or PBS). There was minimal post-operational mortality that was not significantly different between animal groups. Thus, bleomycin injury to the lung caused more severe morbidity that manifested in weight loss than pulmonary CCL18 overexpression.

Histologically, instillation of AdV-CCL18 but not AdV-NULL or PBS caused perivascular and peribronchial lymphocytic infiltration (FIG. 1A,B). The T lymphocytic nature of these cells was confirmed by immunohistochemical staining of lung sections for CD3, CD4, and CD8. Instillation of bleomycin caused the characteristic diffuse interstitial fibrosing alveolitis (FIG. 1C,D).

The combined effect of CCL18 overexpression and bleomycin injury exceeded the effect of each factor alone. It manifested in greater destruction of alveolar architecture, more pronounced perivascular and peribronchial infiltration, and massive lymphocytic accumulation not only in the peribronchial and perivascular areas, but also in the interstitium (FIG. 1E,F). Immunohistochemical analyses for CD3, CD4, CD8, and TCR confirmed such infiltration patterns (an example for CD4+ cells is shown in FIG. 1G-I).

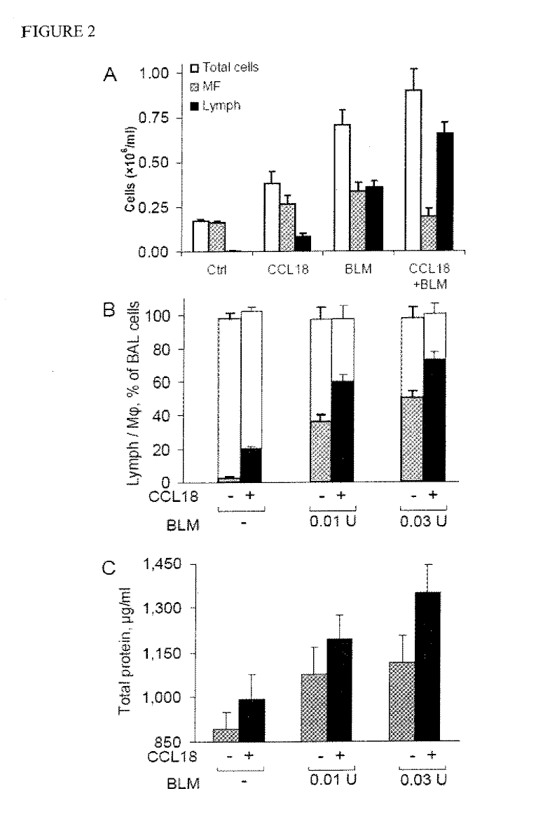

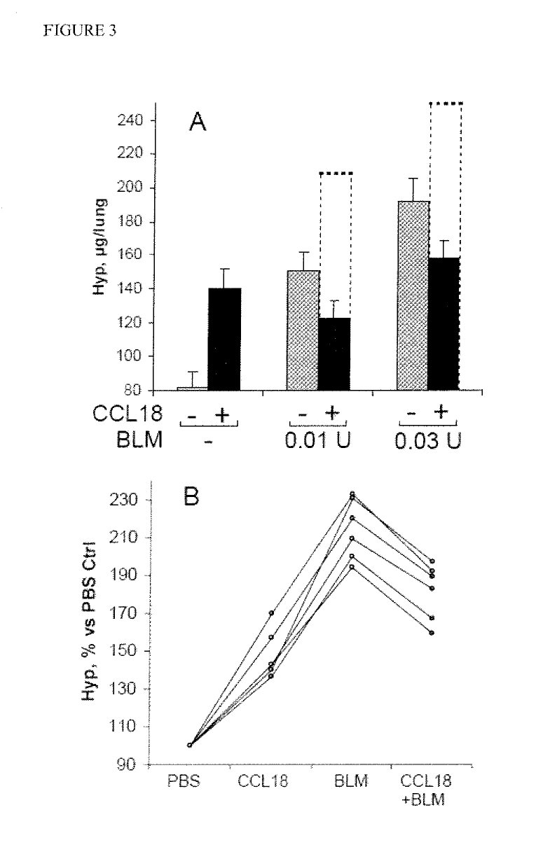

Overexpression of CCL18 and bleomycin injury had an additive effect on T cell content in BAL and total protein concentration in lung homogenates (FIG. 2). Flowcytometric analyses of BAL cells revealed increases in T lymphocytes in CCL18 overexpressing mice as previously reported (see FIG. 3 in ref. 11). Consistent with previous reports (11), gating on CD3+CD4+ and CD3+CD8+ cells revealed the following CD41CD8 ratios: 0.97±0.17 in CCL18 overexpressing mice, 1.61±0.22 in mice challenged with bleomycin, and 0.98±0.09 in mice challenged with CCL18 overexpression and bleomycin injury (n=10 in each group).

In all cases, more than 90% of lung cells were immunohistochemically negative for proliferating cell nuclear antigen (PCNA), suggesting that they are not dividing cells and that the observed dynamics of the infiltration is due to trafficking to the lungs (not shown). Immunohistochemically and flowcytometrically, there was minimal (<1%) presence of CD19+ or B220+ cells. These observations suggested that bleomycin injury and overexpression of CCL18 together elicit a more severe proinflammatory effect on the lung than each of these factors alone, particularly manifesting in additive accumulation of T lymphocytes in the lungs.

Some rare regulatory T cells (FoxP3+) were present in the infiltrates immunohistochemically. Flowcytometric analyses of the permeabilized BAL T cells revealed that 8.94±1.04% of CD4+CD25+FoxP3+ were present in CCL18 overexpressing mice, whereas 8.48±1.22% of such cells were present in mice overexpressing CCL18 in combination with bleomycin injury (n=5 in each group, p>0.05). Separate in vitro chemotaxis experiments using Transwell® system were performed to separate CCL18-responding (lower chamber) from nonresponding (upper chamber) human T cells freshly purified from the PBMC population.

Flow cytometry analyses revealed that in both populations of T cells, those that did or did not respond chemotactically to CCL18, the fractions of CD4+CD25+FoxP3+ cells were similar (6.95±1.5% and 7.08±1.3%, respectively, p>0.05). These in vivo data from animal models and in vitro data obtained with human purified T cells suggest that CCL18, alone or in combination with bleomycin, is not a selective attractor of regulatory T cells.

### Example 6

### Collagen Production and Accumulation in Pulmonary Fibrosis Models

Experiments tested whether CCL18 overexpression and bleomycin in combination would enhance pulmonary fibrosis. In contrast to the additive effect of CCL18 overexpression and injury with bleomycin on the levels of T lymphocytes accumulating in the lungs, the CCL18 overexpression had a partially neutralizing effect on bleomycin-induced collagen accumulation (FIG. 3).

Consistent with previous observations (11), overexpression of CCL18 by itself caused a moderate increase in total pulmonary hydroxyproline content, compared to the increases caused by bleomycin alone (FIG. 3A). However, the combined effect of CCL18 overexpression and bleomycin injury [black bars in the BLM groups in FIG. 3A] was below the expected additive effect of these two factors (unfilled bars, dashed outlines in FIG. 3A). Moreover, the combined effect of CCL18 overexpression and bleomycin injury on collagen accumulation [black bars in the BLM groups in FIG. 3A] was significantly lower than the effect of bleomycin injury alone (grey bars). This observation was made in six independent experiments, with consistent results (FIG. 3B).

An average decrease in total lung Hyp was 33.4±3.9%, based on six independent experiments shown in FIG. 3B. Thus, CCL18 overexpression caused T cell-dependent (11) mild pulmonary fibrosis in an otherwise healthy lung (11), yet it partially protected against severe fibrotic injury caused by bleomycin (FIG. 3). This effect was observed at different non-saturating concentrations of bleomycin (FIG. 3A) and at different times, on day 21 as shown in FIG. 3, and on day 28.

### Example 7

### Determination of the Mechanisms for the Paradoxical Regulation of Collagen Levels in the Combined CCL18 Overexpression and Bleomycin Injury Model

In considering the possible mechanisms that may be involved in the unexpected regulation of collagen levels in the lungs in the combined CCL18 overexpression and bleomycin injury model, the present inventors focused on well known regulators of connective tissue homeostasis, metalloproteinases MMP-2 and MMP-9 (reviewed in 29-32) and major cytokines known to be involved in regulation of inflammation and fibrosis TGF-0, IL-13, TNF-α, IFN-γ, MCP-1 (CCL2) (reviewed in 33) as possible regulators of the observed dynamics in collagen levels.

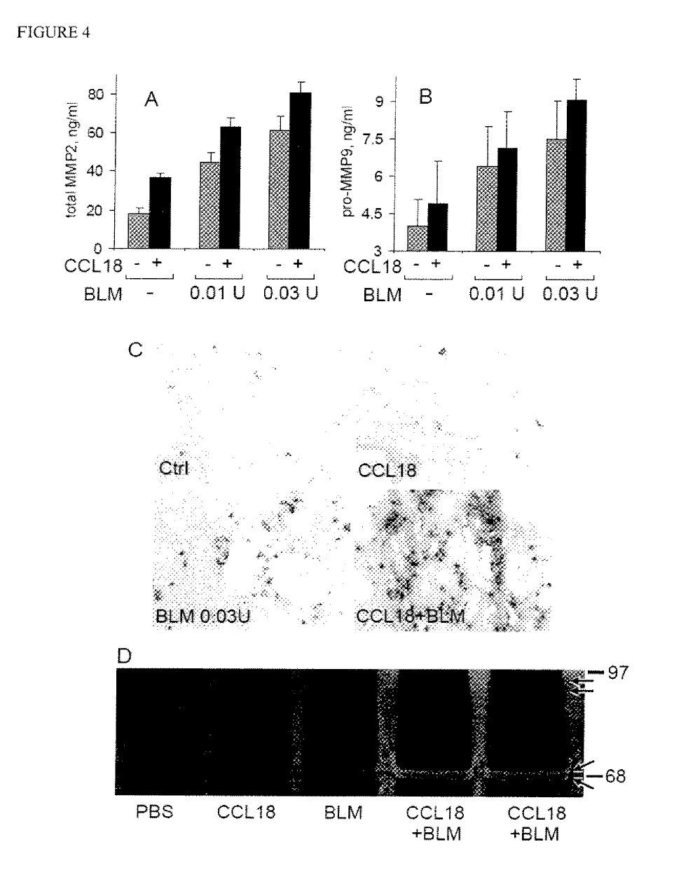

Levels of MMP2 and MMP9 are generally elevated in fibrotic lung diseases, these metalloproteinases are known to contribute both pro- and antifibrotically (29-32). The ELISA assays showed an additive effect of CCL18 overexpression on the levels of pro- and active MMP2 and pro-MMP9 in the lung homogenates (FIG. 4A,B). This additive effect was confirmed immunohistochemically for total (pro- and active) MMP9 (FIG. 4C). Zymographic analyses also revealed an increase in gelatinase activity in the combined CCL18 overexpression and bleomycin injury group (FIG. 4D).

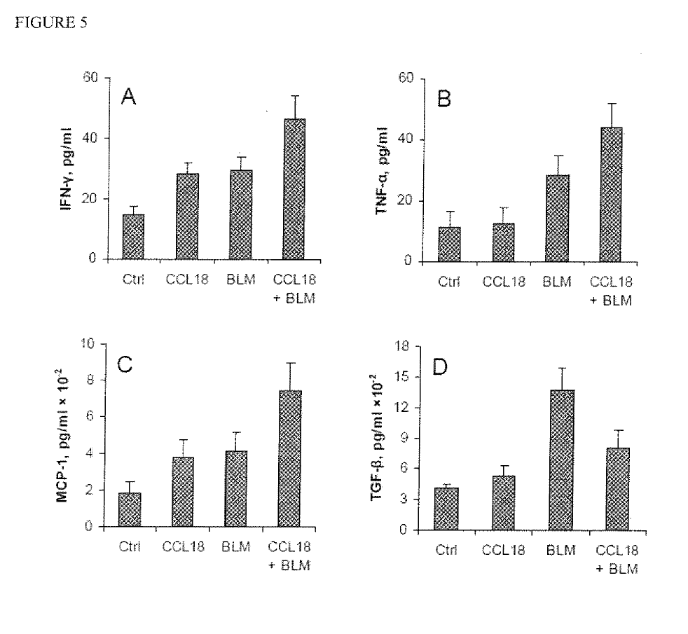

The levels of total TGF-β and IL-13 measured by ELISA varied insignificantly between the groups (p>0.5, Student's t-test, data not shown), similar to our previous report (11). However, overexpression of CCL18 and bleomycin injury additively upregulated the pulmonary levels of IFN-γ, TNF-α, and MCP-1 (FIG. 5A). Mediators IFN-γ and TNF-α have been shown to have both pro- and antifibrotic effects in vivo, with the profibrotic effects being secondary to inflammation, whereas these two factors in vitro are potent inhibitors of collagen production (reviewed in 33).

Thus, the increased levels of these cytokines provide a possible explanation for the decrease in collagen accumulation in the combined CCL18 overexpression and bleomycin injury model. Reciprocally, the levels of a known profibrotic regulator, active TGF-β, in the combined injury model were lower than in mice subjected to bleomycin injury alone (FIG. 5D), which mirrored the changes in hydroxyproline (see FIG. 3).

These observations suggested that multiple mediators, metalloproteinases and cytokines, are involved in the observed regulation of collagen levels in the combined CCL18 overexpression and bleomycin injury model.

### Example 8

### Effect of Lymphocyte Depletion and Camp Neutralization on Collagen Accumulation in the Lungs

It was Determined if CCL18 by itself was sufficient to cause the changes in collagen accumulation, or whether there is a need for pulmonary T cells in mediating the observed effects.

Real-time PCR and ELISA showed that mice treated with anti-lymphocyte serum (ALS) before the administration of AdV-CCL18 did not affect CCL18 expression in the lung. However, there was complete abrogation of the perivascular and peribronchial infiltration and collagen accumulation consistent with previous reports (11). In addition, consistent with other previous reports (22,23,25), treatment with ALS did not have a significant effect on collagen accumulation in the bleomycin injury model (p>0.05, Student's t-test).

In the combined CCL18 overexpression and bleomycin injury model treated with ALS, pulmonary levels of collagen did not differ from the bleomycin injury alone treated with ALS (205.8±14.1 pg/lung vs 199±12.3 pg/lung, respectively, p>0.05, Student's t-test, data not shown). Thus, the effect of bleomycin injury alone on collagen accumulation appears to be T cell-independent, whereas depletion of T cells eliminates the effect of CCL18 overexpression on collagen accumulation the normal or bleomycin-damaged lung.

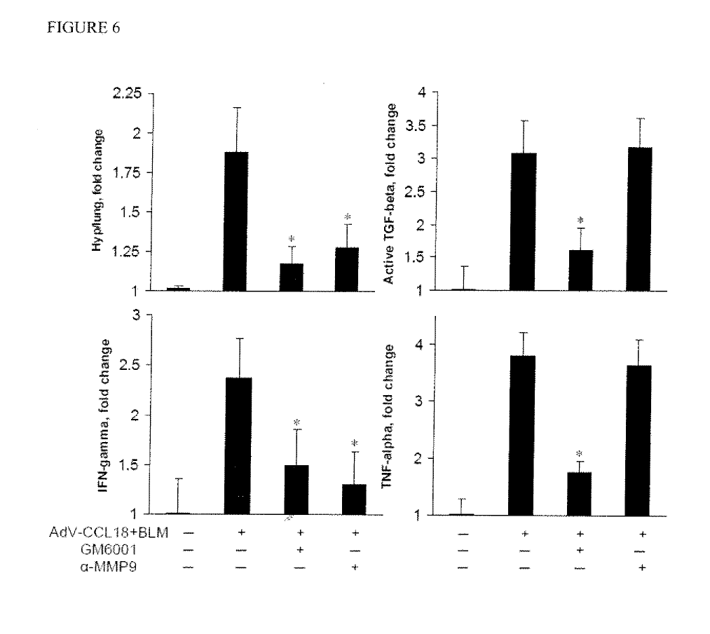

The contribution of MMPs to tissue fibrosis is complex (29-32). The changes in the levels of MMPs (see FIG. 4) were determined in regard to the regulation of collagen and cytokine levels. Administration of GM6001, a broad-spectrum pharmacological MMP inhibitor, significantly abrogated pulmonary levels of hydroxyproline, active TGF-β, TNF-α and IFN-γ in the combined injury model (FIG. 6), suggesting a central involvement of MMPs in inflammatory and fibrotic processes. Administration of neutralizing anti-MMP9 antibody had no effect on the levels of active TGF-β or TNF-α but abrogated the levels of IFN-γ and further abrogated the levels of hydroxyproline, suggesting that MMP2 is likely to be involved in regulation of TGF-β and TNF-αlevels. Thus, MMP2 and MMP9 contribute to the regulation of collagen and cytokine levels in the combined injury model.

### Example 9

### Recombinant Adenovirus-Mediated CCL18 Gene Delivery and Bleomycin-Induced Drug Injury

Adenovirus-mediated gene delivery combined with bleomycin-induced injury to the lung is conducted according to established methods (22,34,35).

Mice are instilled with AdV-CCL18(11), and then at the peak of CCL18 production (day 7), a second intratracheal instillation is performed with either PBS (as a control) or bleomycin, in lower (0.01 U/mouse) or higher (0.03 U/mouse) dose to induce lung inflammation and fibrosis. The selected doses of bleomycin alone were found to be insufficient to achieve a plateauing effect on lung inflammation and fibrosis in preliminary experiments.

The bleomycin challenge but not instillation of PBS caused a statistically significant transient loss of body weight on days 5-20 following the second intratracheal instillation, independent of the nature of the agent used for the first instillation (AdV-CCL8, AdV-NULL, or PBS). Thus, bleomycin injury to the lung causes more severe morbidity manifested in weight loss than pulmonary CCL18 overexpression. The combined effect of CCL18 overexpression and bleomycin injury exceeded the effect of each factor alone on the severity of histological changes. It manifested in greater destruction of alveolar architecture, more pronounced perivascular and peribronchial infiltration, and massive lymphocytic accumulation not only in the peribronchial and perivascular areas, but also in the interstitium (see FIG. 1).

Overexpression of CCL18 and the bleomycin injury had an additive effect on T cell content in the BAL samples and total protein concentration in lung homogenates (see FIG. 2). Thus, overexpression of CCL18 and the bleomycin injury together elicit a more severe proinflammatory effect on the lung than each of these factors alone.

In contrast to the additive effect on inflammation, CCL18 overexpression unexpectedly attenuated the severe bleomycin-induced collagen accumulation (see FIG. 3). This finding suggested that although CCL18-induced T lymphocytic infiltration is by itself mildly profibrotic to a healthy lung (11), the very same infiltration may be partially protective against severe fibrosis in a proinflammatory profibrotic setting in the lungs.

Although a consistently reproducible phenomenon (see FIG. 3B), the amplitude of decline in pulmonary collagen level in the combined model compared with bleomycin alone is constitutes about 33.4±3.9% of the normal collagen content. However, it is important to consider that fibrosis correlates with decline in lung function (36), and that the present method provided an approximately 30% increase in pulmonary function in comparison to another therapy that reportedly resulted in only 3% higher forced vital capacity than placebo and which is celebrated as a significant achievement in treating patients with scleroderma lung disease (37).

The present invention shows that further enhancing the antifibrotic regulation in the lungs may be accomplished by therapeutically manipulating the local pulmonary milieu and/or the phenotypes of infiltrating T lymphocytes.

There are sporadic data on antifibrotic effects of T lymphocytes in vitro (38,39) and in vivo (40). The idiopathic pulmonary fibrosis (IPF) patients with a higher percentage of BAL T lymphocytes may be more responsive to treatment (41). In combination with the present findings, these data show that T cell infiltration in patients with pulmonary fibrosis is part of an antifibrotic feedback loop, suggesting that eliminating T cells from the inflamed lung may promote fibrosis and thus have a counterintuitive deleterious effect.

Depletion of lymphocytes with an anti-lymphocyte serum abrogated the perivascular and peribronchial infiltration and collagen accumulation (not shown), consistent with our previous report (11). The present inventors have found that systemic antibody-mediated depletion of T lymphocytes completely abrogates the effects of CCL18 overexpression in the lungs (11).

Similarly, treatment with antilymphocyte serum in the combined CCL18 overexpression and bleomycin injury model did abrogate the partial protective effect of CCL18 overexpression in the combined model in immunocompetent mice; pulmonary levels of collagen in the T lymphocyte-depleted animals that overexpressed CCL18 and were challenged with bleomycin did not differ from the immunocompetent mice challenged with bleomycin alone.

Accordingly, T lymphocytes may be therapeutically modulated to act antifibrotically, instead of being targeted and eliminated from the lungs.

The present inventors have investigated the mechanism of the paradoxical regulation of the collagen accumulation in the combined CCL18 overexpression and bleomycin injury model. One possible mechanism may involve regulatory T cell; however, no differences between the studied groups of animals were found in the content of CD4+CD25+FoxP3+ cells. Also, in vitro chemotaxis assays revealed that CCL18 did not selectively attract such regulatory T cells. Together, these observations suggested that T regulatory T cells explain the differences in collagen accumulation between animal groups.

Metalloproteinases MMP-2 and MMP-9 are well known regulators of connective tissue homeostasis that are involved in lung inflammation and fibrosis and act dually, proteolytically and non-proteolytically, in a complex concentration-dependent fashion (29-32). Depending on the specifics of the inflammatory milieu and local concentration of MMPs, their effects may be either pro- or antifibrotic.

These data show that overexpression of CCL18 and injury with bleomycin acted additively on MMP2 and MMP9 levels and activity in the lungs. As part of addressing the observed mechanism, the levels of pro- and antifibrotic cytokines were measured. The levels of proinflammatory cytokines TNF-α and IFN-γ were additively upregulated by CCL18 overexpression and the bleomycin injury (see FIG. 5A). These two factors are known to have complex effect on tissue fibrosis, including their direct and indirect regulation of collagen production by fibroblasts (reviewed in 33).

One line of evidence, in in vitro studies suggests that they are potent direct inhibitors of collagen production in fibroblasts. In contrast, in some animal models, each of these cytokines indirectly facilitated fibrosis through inflammation-dependent mechanisms (reviewed in 33). The results on the elevation of these potentially antifibrotic cytokines shown in FIGS. 5 and 6 are consistent with their antifibrotic action, leading to decline in collagen levels in the combined injury model (see FIG. 3).

Reciprocally, the levels of active TGF-β declined in the combined injury model (see FIG. 5B), also consistent with the changes in collagen accumulation (see FIG. 3). Thus, the levels of the profibrotic factor, active TGF-β, mirrored those of collagen, whereas the levels of potentially antifibrotic factors TNF-α and IFN-γ changed reciprocally, in agreement with the attenuation of the collagen content in the combined CCL18 overexpression and bleomycin injury model.

Therefore, numerous factors are involved in the observed paradoxical regulation of collagen accumulation in the combined CCL18 overexpression and bleomycin injury model. Although TGF-β is a central mediator of bleomycin-induced lung fibrosis, increases in the total levels of this powerful profibrotic cytokine are difficult to detect because of the overall high basal level of inactive TGF-β (e.g. see FIG. 5C in ref. 42). Significant changes in total pulmonary TGF-β in the studied models could not be detected. The present inventors also found no significant changes in the levels of a potent profibrotic cytokine IL-13.

Neutralization of MMPs with a broad-spectrum inhibitor GM6001 further attenuated the decline in collagen accumulation and the levels of the profibrotic and proinflammatory cytokines in animals with the combined CCL18 overexpression and bleomycin injury (see FIG. 6), confirming a significant role for MMPs and a potential for therapeutic modulation of these enzymes in lung fibrosis. A selective neutralization of MMP9 with a specific neutralizing antibody attenuated the levels of collagen and IFN-7 but not TNF-α and active TGF-61, indicating that MMP2 may be required for regulation of levels of the two latter cytokines.

The novelty of these results is that the CCL18-mediated T lymphocytic infiltration is profibrotic in the otherwise healthy lungs but it is unexpectedly partially antifibrotic when superimposed on a second profibrotic injury (bleomycin). It also appears that the regulation of CCL18-mediated pulmonary inflammation and fibrosis is complex and occurs through mechanisms that involve T lymphocytic infiltration, matrix metalloproteinases, and profibrotic and proinflammatory cytokines. The simplistic approach to developing new antifibrotic therapies in which pulmonary T lymphocytes are selectively targeted to diminish the degree of fibrosis should be reconsidered, as these cells play a partially protective role and could be phenotypically modulated to act even more antifibrotically.

### LIST OF REFERENCES

The following references are incorporated by reference in their entirety:
1. Luzina I G, Atamas S P, Wise X, Wigley F M, Xiao H Q, White B. Gene expression in bronchoalveolar lavage cells from scleroderma patients. Am J Respir Cell Mol Biol 2002, 26:549-57.
2. Atamas S P, Yurovsky V V, Wise K, Wigley F M, Goter Robinson C J, Henry P, Alms W J, White B. Production of type 2 cytokines by CD8+ lung cells is associated with greater decline in pulmonary function in patients with systemic sclerosis. Arthritis Rheum 1999, 42:1168-78.
3. Pardo A, Smith K M, Abrams J, Coffman R, Bustos M, McClanahan T K, Grein J, Murphy E L, Zlotnik A, Selman M. CCL18/DC-CK-1/PARC up-regulation in hypersensitivity pneumonitis. J Leukoc Biol 2001, 70:610-6.
4. Prasse A, Pechkovsky D V, Toews G B, Jungraithmayr W, Kollert F. Goldmann T, Vollmer E, Muller-Quernheim J, Zissel G. A vicious circle of alveolar macrophages and fibroblasts perpetuates pulmonary fibrosis via CCL18. Am J Respir Crit Care Med 2006, 173:781-92
5. Mrazek F, Sekerova V, Drabek J, Kolek V, du Bois R M, Petrek M. Expression of the chemokine PARC mRNA in bronchoalveolar cells of patients with sarcoidosis. Immunol Lett 2002, 84:17-22.
6. de Nadai P, Charbonnier A S, Chenivesse C, Senechal S, Fournier C, Gilet J, Vorng H, Chang Y, Gosset P, Wallaert B, Tonne! A B, Lassalle P, Tsicopoulos A. Involvement of CCL18 in allergic asthma. J Immunol 2006, 176:6286-93,
7. Zou J, Young S, Zhu F, Gheyas F, Skeans S, Wan Y, Wang L, Ding W, Billah M, McClanahan T, Coffman R L, Egan R, Umland S. Microarray profile of differentially expressed genes in a monkey model of allergic asthma. Genome Biol 2002, 3:0020.1-0020.13.
8. Kodelja V, Muller C, Politz 0, Hakij N, Orfanos C E, Goerdt S. Alternative macrophage activation-associated CC-chemokine-1, a novel structural homologue of macrophage inflammatory protein-1a with a Th2-associated expression pattern. J Immunol 1998, 160:14118.
9. Hieshima K, Imai T, Baba M, Shoudai K, Ishizuka K, Nakagawa T, Tsuruta S, Takeya M, Sakaki Y, Takatsuki K, Miura R, Opdenakker G, Van Damme J, Yoshie 0, Nomiyama H. A novel human CC chemokine PARC that is most homologous to macrophage-inflammatory protein-1 a/LD78a and chemotactic for T lymphocytes, but not for monocytes. J Immunol 1997, 159:1140-9.
10. Guan P, Burghes A H, Cunningham A, Lira P, Brissette W H, Neote K, McColl S R. Genomic organization and biological characterization of the novel human CC chemokine DC-CK1/PARC/MIP-4/SCYA18. Genomics 1999, 56:296-302.
11. Luzina I G, Papadimitriou J C, Anderson R, Pochetuhen K, Atamas S P. Induction of prolonged infiltration of T lymphocytes and transient T lymphocyte-dependent collagen deposition in mouse lungs following adenoviral gene transfer of CCU 8. Arthritis Rheum 2006, 54:2643-55.
12. Atamas S P, Luzina I G, Choi J, Tsymbalyuk N, Carbonetti N H, Singh I S, Trojanowska M, Jimenez S A, White B. Pulmonary and activation-regulated chemokine stimulates collagen production in lung fibroblasts. Am J Respir Cell Mol Biol 2003, 29:743-9.
13. Luzina I G, Tsymbalyuk N, Choi J, Hasday J D, Atamas S P. CCU 8-stimulated upregulation of collagen production in lung fibroblasts requires SP1 signaling and basal Smad3 activity. J Cell Physiol 2006, 206:221-8.
14. Luzina I G, Highsmith K, Pochetuhen K, Nacu N, Rao J N, Atamas S P. PKCalpha mediates CCL18-stimulated collagen production in pulmonary fibroblasts. Am J Respir Cell Mol Biol 2006, 35:298-305.
15. Gordon S. Alternative activation of macrophages. Nat Rev Immunol 2003, 3:23-5.
16. Goerdt S, Politz 0, Schledzewski K, Birk R, Gratchev A, Guillot P, Hakiy N, Klemke C D, Dippel E, Kodeljia V, Orfanos C E. Alternative versus classical activation of macrophages. Pathobiology 1999, 67.222-6.
17. Gratchev A, Guillot P, Hakiy N, Politz 0, Orfanos C E, Schledzewski K, Goerdt S. Alternatively activated macrophages differentially express fibronectin and its splice variants and the extracellular matrix protein 13IG-H3. Scand J Immunol 2001, 53:386-92.
18. Song E, Ouyang N, Horbelt M, Antus B, Wang M, Exton M S. Influence of alternatively and classically activated macrophages on fibrogenic activities of human fibroblasts. Cell Immunol 2000, 204:19-28.
19. Luzina I G, Atamas S P, Wise K, Wigley F M, Choi J, Xiao H Q, White B. Occurrence of an activated, profibrotic pattern of gene expression in lung CD8+ T cells from scleroderma patients. Arthritis Rheum 2003, 48:2262-74.
20. Atamas S P, Luzina I G, Dai H, Wilt S G, White B. Synergy between CD40 ligation and IL-4 on fibroblast proliferation involves IL-4 receptor signaling. J Immunol 2002, 168:1139-45.
21. Sempowski G D, Chess P R, Phipps R P. CD40 is a functional activation antigen and B7independent T cell costimulatory molecule on normal human lung fibroblasts. J Immunol 1997, 158:4670-7.
22. Huaux F, Liu T, McGarry B, Ullenbruch M, Xing Z, Phan S H. Eosinophils and T lymphocytes possess distinct roles in bleomycininduced lung injury and fibrosis. J Immunol 2003, 171:547081.
23. Helene M, Lake-Bullock V, Zhu J, Hao H, Cohen D A, Kaplan A M. T cell independence of bleomycin-induced pulmonary fibrosis. J Leukoc Biol 1999, 65:187-95.
24. Suzuki N, Ohta K, Horiuchi T, Takizawa H, Ueda T, Kuwabara M, Shiga J, Ito K. T lymphocytes and silica-induced pulmonary inflammation and fibrosis in mice. Thorax 1996, 51:1036-42.
25. Janick-Buckner D, Ranges G E, Hacker M P. Effect of cytotoxic monoclonal antibody depletion of I-lymphocyte subpopulations on bleomycin-induced lung damage in C57BL/6J mice. Toxicol Appl Pharmacol 1989, 100:474-84.
26. Hao Z, Hampel B, Yagita H, Rajewsky K. T cell-specific ablation of Fas leads to Fas ligandmediated lymphocyte depletion and inflammatory pulmonary fibrosis. J Exp Med 2004, 199:1355-65.
27. Okazaki T, Nakao A, Nakano H, Takahashi F, Takahashi K, Shimozato 0, Takeda K, Yagita H, Okumura K. Impairment of bleomycin-induced lung fibrosis in CD28-deficient mice. J Immunol 2001, 167:1977-81.
28. Bonniaud P, Margetts P J, Ask K, Flanders K, Gauldie J, Kolb M. TGF-8 and Smad3 signaling link inflammation to chronic fibrogenesis. J Immunol 2005, 175:5390-5.
29. Corbel M, Belleguic C, Boichot B, Lagente V. Involvement of gelatinases (MMP-2 and MMP9) in the development of airway inflammation and pulmonary fibrosis. Cell Biol Toxicol 2002, 18:51-61.
30. Chakrabarti S, Patel K D. Matrix metalloproteinase-2 (MMP-2) and MMP-9 in pulmonary pathology. Exp Lung Res 2005, 31:599-621.
31. Tan R J, Fattman C L, Niehouse L M, Tobolewski J M, Hanford L E, Li Q, Monzon F A, Parks W C, Oury T D. Matrix metalloproteinases promote inflammation and fibrosis in asbestos-induced lung injury in mice. Am J Respir Cell Mol Biol 2006, 35:289-97.
32. Atkinson J J, Senior R M. Matrix metalloproteinase-9 in lung remodeling. Am J Respir Cell Mol Biol 2003, 28:12-24.
33. Atamas S P, White B. Cytokine regulation of pulmonary fibrosis in scleroderma. Cytokine Growth Factor Rev 2003, 14:537-50.
34. Fujita M, Shannon J M, Morikawa 0, Gauldie J, Nara N, Mason R J. Overexpression of tumor necrosis factor-alpha diminishes pulmonary fibrosis induced by bleomycin or transforming growth factor-beta. Am J Respir Cell Mol Biol 2003, 29:669-76.
35. Bonniaud P, Martin G, Margetts P J, Ask K, Robertson J, Gauldie J, Kolb M. Connective tissue growth factor is crucial to inducing a profibrotic environment in “fibrosis-resistant” BALD/c mouse lungs. Am J Respir Cell Mol Biol 2004, 31:5106.
36. Martinez F J, Flaherty K. Pulmolnary function testing in idiopathic interstitial pneumonias. Proc Am Thorac Soc 2006, 3:315-21.
37. Tashkin D P, Elashoff R, Clements P J, Goldin J, Roth M D, Furst D E, Arriola E, Silver R, Strange C, Bolster M, Seibold J R, Riley D J, Hsu V M, Varga J, Schraufnagel D E, Theodore A, Simms R, Wise R, Wigley F, White B, Steen V, Read C, Mayes M, Parsley E, Mubarak K, Connolly MK, Golden J, Olman M, Fessler B, Rothfleld N, Metersky M; Scleroderma Lung Study Research Group. Cyclophosphamide versus placebo in scleroderma lung disease. N Engl J Med 2006, 354:2655-66.
38. Chizzolini C, Pare! Y, De Luca C, Tyndall A, Akesson A, Scheja A, Dayer J M. Systemic sclerosis Th2 cells inhibit collagen production by dermal fibroblasts via membrane-associated tumor necrosis factor alpha. Arthritis Rheum 2003, 48:2593-604.
39. Chizzolini C, Rezzonico R, Ribbens C, Burger D, Wollheim F A, Dayer J M. Inhibition of type I collagen production by dermal fibroblasts upon contact with activated T cells: different sensitivity to inhibition between systemic sclerosis and control fibroblasts. Arthritis Rheum 1998, 41:203947.
40. Corsini F, Luster M I, Mahler J, Craig W A, Blazka M E, Rosenthal G J. A protective role for T lymphocytes in asbestos-induced pulmonary inflammation and collagen deposition. Am J Respir Cell Viol Biol 1994, 11:531-9.
41. Fireman E, Vardinon N, Burke M, Spizer S, Levin S, Endler A, Stav D, Topilsky M, Mann A, Schwarz Y, Kivity S, Greif J. Predictive value of response to treatment of T-lymphocyte subpopulations in idiopathic pulmonary fibrosis. Eur Respir J 1998, 11:706-11.
42. Huang M, Sharma S, Zhu LX, Keane M P, Luo J, Zhang L, Burdick M D, Lin YQ, Dohadwala M, Gardner B, Batra R K, Strieter R M, Dubinett S M. IL-7 inhibits fibroblast TGF-beta production and signaling in pulmonary fibrosis, J Clin Invest 2002, 109:931-7.

What is claimed is:

1. A method of treating severe or rapidly progressing pulmonary fibrosis in a subject in need of a treatment thereof, the method comprising increasing the activity of pulmonary and activation-regulated chemokine (CCL18) in the lungs of the subject, whereby increasing CCL18 activity modulates the activity of at least one antifibrotic factor in the lungs of the subject.

2. The method of claim 1, wherein increasing the activity comprises increasing the expression of a peptide with CCL18 activity.

3. The method of claim 2, wherein the peptide is CCL18.

4. The method of claim 3, wherein the CCL18 is native to the subject.

5. The method of claim 4, wherein the CCL18 is not native to the subject.

6. The method of claim 3, wherein increasing the expression of CCL18 comprises transfection.

7. The method of claim 6, wherein transfection comprises a viral vector or a non-viral vector.

8. The method of claim 6, wherein the transfection comprises a technique selected from the group consisting of magnetotransfection, cationic lipid-based delivery, electroporation and combinations thereof.

9. The method of claim 1, wherein the severe or rapidly progressing pulmonary fibrosis is associated with a disorder selected from the group consisting of scleroderma lung disease, saracoidosis, Wegener's granulomatosis, infections, asbestosis, ionizing radiation exposure, lupus, rheumatoid arthritis, hypersensitivity pneumonitis, nonspecific interstitial pneumonitis, Hamman-Rich Syndrome, diffuse fibrosing alveolitis, idiopathic pulmonary fibrosis, idiopathic pulmonary fibrosis and combinations thereof.

10. The method of claim 1, wherein the at least one antifibrotic factor in the lungs of the subject is selected from the group consisting of matrix metalloproteinase-2 (MMP2), matrix metalloproteinase-9 (MMP9), tumor necrosis factor alpha (TNF-α), interleukin-8 (TL-8), interleukin-1 (IL-1), T cells, B cells, natural killer (NK) cells, interferon gamma (IFN-γ), interferon alpha (IFN-α), and combinations thereof.

11. The method of claim 1, wherein the severe or rapidly progressing pulmonary fibrosis is associated with tissue injury.

12. The method of claim 11, wherein the tissue injury is caused by an injury-inducing agent selected from the group consisting of an anticonvulsant drug, an antipsychotic drug, an antidepressant drug, an anti-inflammatory drug, an antimetabolic drug, an antimicrobial drug, biologic response modifiers, a cardiovascular drug, a chemotherapeutic drug, an immunosuppressive drug, and combinations thereof.

13. The method of claim 12, wherein the antimicrobial drug is selected from the group consisting of nitrofurantoin, sulfasalazine, tetracycline, minocycline, sulfonamides, parpa-aminosalicyclic acid, ethambutol, ampicillin, cephalosporin, and a combination thereof.

14. The method of claim 12, wherein the cardiovascular drug is selected from the group consisting of amiodarone, angiotensin-converting enzyme (ACE) inhibitor, and a combination thereof.

15. The method of claim 12, wherein the chemotherapeutic drug is selected from the group consisting of bleomycin, mitomycin-C, busulfan, cyclophosphamide, nitrosourea, procarbazine, melphalan, paclitaxel, and a combination thereof.

16. The method of claim 15, wherein the chemotherapeutic drug is bleomycin.

17. A method of treating severe or rapidly progressing pulmonary fibrosis in a subject in need of a treatment thereof, the method comprising administering to the subject a means for increasing the activity of pulmonary and activation-regulated chemokine (CCL18) in the lungs of the subject, whereby increasing CCL18 activity modulates the activity of at least one anti fibrotic factor in the lungs of the subject.

18. A method of screening a compound that may alter the progression of severe or rapidly progressing pulmonary fibrosis, the method comprising
a) administering an injury-inducing agent to a control and test population of cells, wherein the injury-inducing agent is known to produce severe or rapidly progressing pulmonary fibrosis, and
b) administering a test procedure to the test population of injured cells,
c) observing test level activities of pulmonary and activation-regulated chemokine (CCL18)
d) comparing the test activity levels of CCL18 with a standard activity level of CCL18, wherein the standard activity level of CCL18 are established in the control population of injured cells,
wherein an increase in the activity levels of CCL18 in the test population over the standard CCL18 activity levels indicates that the test procedure may be capable of altering the progression of severe or rapidly progressing pulmonary fibrosis.

19. The method of claim 18, wherein the test procedure is suspected of being able to increase the activity of pulmonary and activation-regulated chemokine (CCL18) in cell populations.

20. The method of claim 18, wherein the activity levels of CCL18 are measured using the activity of at least one antifibrotic factor.
